(19) United States
(12) Patent Application Publication
(10) Pub. No.: US 2026/0144918 A1
(43) Pub. Date: May 28, 2026

(54) FILTER DEVICE

(71) Applicant: TERUMO KABUSHIKI KAISHA, Tokyo (JP)

(72) Inventor: Naoki IIDA, Fujinomiya-shi Shizuoka (JP)

(21) Appl. No.: 19/419,862

(22) Filed: Dec. 15, 2025

Related U.S. Application Data
(63) Continuation of application No. PCT/JP2024/021794, filed on Jun. 17, 2024.

(30) Foreign Application Priority Data
Jun. 16, 2023 (JP) 2023099348

(51) Int. Cl. A61M 1/02; B04B 3/00; B04B 5/04

(52) U.S. Cl. CPC A61M 1/0281; A61M 1/0272; B04B 3/00; B04B 5/0442; A61M 2202/0439; A61M 2205/125; B04B 2005/0478

(57) ABSTRACT

A filter device according to the present invention is to be mounted on a centrifugal separator and includes: a first space into which blood flows; a second space from which the blood flows out; a partition wall separating the first space and the second space; a communication port establishing communication between the first space and the second space; and a white-blood-cell removal filter disposed in the second space. The first space and the second space are arranged in a first direction intersecting a centrifugal direction, and the inlet port and the communication port are located between a center line along the first direction and a rotation center of the centrifugal separator.

### CROSS-REFERENCE TO RELATED APPLICATIONS

The present disclosure is a continuation of and claims benefit to PCT/JP2024/021794 filed on Jun. 17, 2024, entitled “FILTER DEVICE” which claims priority to Japanese Patent Application No. 2023-099348, filed on Jun. 16, 2023. The entire disclosure of the applications listed above are hereby incorporated herein by reference, in their entireties, for all that they teach and for all purposes.

### BACKGROUND

The present disclosure relates to a filter device.

Japanese U.S. Pat. No. 5,223,006 B2 describes a blood bag system and a centrifugal transfer device (e.g., centrifugal separator). The blood bag system accommodates blood. The centrifugal separator centrifuges the blood accommodated in the blood bag system. The centrifuged blood is used for blood transfusion.

### BRIEF SUMMARY

It is preferable that blood to be transfused does not contain white blood cells. Recently, a filter device capable of more suitably removing white blood cells is desired.

It is an object of the present disclosure to solve the problem described above.

One aspect of the present disclosure is (1) a filter device to be mounted on a centrifugal separator, the filter device including: a first space which is formed in a housing and into which blood flows through an inlet port; a second space which is formed in the housing and from which the blood flows out through an outlet port; a partition wall that separates the first space and the second space; a communication port that establishes communication between the first space and the second space; and a white-blood-cell removal filter that is disposed in the second space and that removes a white blood cell contained in the blood, wherein the first space and the second space are arranged in a first direction intersecting a centrifugal direction in which a centrifugal force is applied, and the inlet port and the communication port are located between a center line of the housing along the first direction and a rotation center of the centrifugal separator.

With this configuration, white blood cells can be suitably removed from blood.

Aspect (2) describes the filter device according to aspect (1), in which the outlet port may be located in the centrifugal direction relative to the center line.

With this configuration, the blood can be smoothly taken out from the outlet port only by operating the centrifugal separator.

Aspect (3) describes the filter device according to aspect (1) or aspect (2), in which the inlet port may be located on one side of the first space in the first direction, and the communication port may be located on another side of the first space in the first direction.

This configuration makes it possible to prevent blood from which white blood cells have not been removed by centrifugation from reaching the second space.

Aspect (4) describes the filter device according to any one of aspects (1) to (3) provided above, in which the first space may include a curved wall, and the curved wall may be located in the centrifugal direction relative to the center line and located on one side of the first space.

This configuration makes it possible to prevent backflow of blood toward the inlet port.

Aspect (5) describes the filter device according to any one of aspects (1) to (4), in which the communication port may be located on one side of the second space in the first direction, and the outlet port may be located on another side of the second space in the first direction.

This configuration makes it possible to more reliably remove white blood cells by the white-blood-cell removal filter.

Aspect (6) describes the filter device according to any one of aspects (1) to (5), in which the first space may be provided with a meandering flow channel for allowing the blood flowing in through the inlet port to meander.

With this configuration, white blood cells can be suitably removed from blood.

This configuration makes it possible to prevent blood from which white blood cells have not been removed by centrifugation from reaching the second space.

One aspect of the present disclosure is (7) a filter device, comprising: a housing comprising a length extending along a first direction from a first end of the filter device to a second end of the filter device, the housing comprising a width extending along a second direction from a first side of the filter device to a second side of the filter device, wherein a center of the width defines a center line extending along the first direction bisecting the width between the first side of the filter device and the second side of the filter device, and wherein the housing further comprises: a first space disposed inside the housing adjacent the first end of the filter device, the first space spanning across the center line, wherein the first space comprises a curved interior wall arranged adjacent the second side of the filter device; a second space disposed inside the housing adjacent the second end of the filter device, the second space spanning across the center line; a partition wall arranged between the first space and the second space, wherein the partition wall separates the first space from the second space; an inlet port arranged on the first side of the filter device, wherein the inlet port is in fluid communication with the first space, and wherein the inlet port comprises an inlet flow direction extending toward the curved interior wall of the first space; a communication port arranged in the partition wall, wherein the communication port fluidly interconnects the first space and the second space; and an outlet port arranged on the second side of the filter device, wherein the outlet port is in fluid communication with the second space; wherein, when a centrifugal force is applied to the filter device in the second direction, a blood flow path is defined between the inlet port, the curved interior wall, the communication port, and the outlet port.

Aspect (8) describes the filter device according to aspect (7) wherein the inlet flow direction extends into the first space along the second direction. Aspect (9) describes the filter device according to any one of aspects (7) to (8), wherein the outlet port comprises an outlet flow direction that extends out of the second space along the first direction. Aspect (10) describes the filter device according to any one of aspects (7) to (9), wherein the communication port comprises a communication flow direction that extends from the first space into the second space along the first direction. Aspect (11) describes the filter device according to any one of aspects (7) to (10), wherein the outlet port is arranged adjacent the second end of the filter device. Aspect (12) describes the filter device according to any one of aspects (7) to (11), wherein the communication port is arranged closer to the first side of the filter device than the second side of the filter device. Aspect (13) describes the filter device according to any one of aspects (7) to (12), wherein the curved interior wall provides a smooth curved flow transition in the blood flow path at a first wall portion in the first space that is arranged parallel to the second direction to a second wall portion of the first space that is arranged parallel to the first direction. Aspect (14) describes the filter device according to any one of aspects (7) to (13), wherein the first wall portion is arranged on the first side of the filter device adjacent the first end of the filter device. Aspect (15) describes the filter device according to any one of aspects (7) to (14), wherein the second wall portion is arranged on the second side of the filter device adjacent the partition wall. Aspect (16) describes the filter device according to any one of aspects (7) to (15), wherein a majority of the curved interior wall is arranged on the second side of the filter device. Aspect (17) describes the filter device according to any one of aspects (7) to (16), further comprising: a white-blood-cell removal filter disposed inside the second space, wherein the white-blood-cell removal filter comprises a filter medium that is configured to remove white blood cells from a portion of blood flowing therethrough. Aspect (18) describes the filter device according to any one of aspects (7) to (17), wherein the first space further comprises a meandering flow channel that changes a direction of blood flowing along the blood flow path inside the first space before reaching the communication port.

One aspect of the present disclosure is (19) a filter device, comprising: a housing, comprising: a first interior space formed inside the housing, the first interior space comprising an inlet port arranged in fluid communication with the first interior space and a curved interior wall arranged opposite the inlet port; a second interior space formed inside the housing, the second interior space comprising an outlet port arranged in fluid communication with the second interior space; a partition wall arranged between the first interior space and the second interior space, wherein the partition wall separates the first interior space from the second interior space; and a communication port disposed in the partition wall, the communication port arranged in fluid communication with the first interior space and the second interior space; wherein the first interior space and the second interior space are arranged side-by-side in a first direction that is arranged perpendicular to a centrifugal direction, wherein, when a centrifugal force is applied to the filter device in the centrifugal direction, a blood flow path is defined between the inlet port, the curved interior wall, the communication port, and the outlet port.

Aspect (20) describes the filter device according to aspect (19) further comprising: a white-blood-cell removal filter disposed inside the second interior space, wherein the white-blood-cell removal filter comprises a filter medium that is configured to remove white blood cells from a portion of blood flowing through the white-blood-cell removal filter.

According to the present disclosure, it is possible to suitably remove white blood cells from blood.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a perspective view illustrating a centrifugal separator to which a filter device according to embodiments of the present disclosure is mounted.

FIG. 2 is a perspective view illustrating the filter device in accordance with embodiments of the present disclosure.

FIG. 3 is a plan view illustrating an internal structure of the filter device in accordance with embodiments of the present disclosure.

FIG. 4 is a plan view illustrating an internal structure of a filter device according to a first modification.

FIG. 5 is a plan view illustrating an internal structure of a filter device according to a second modification.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

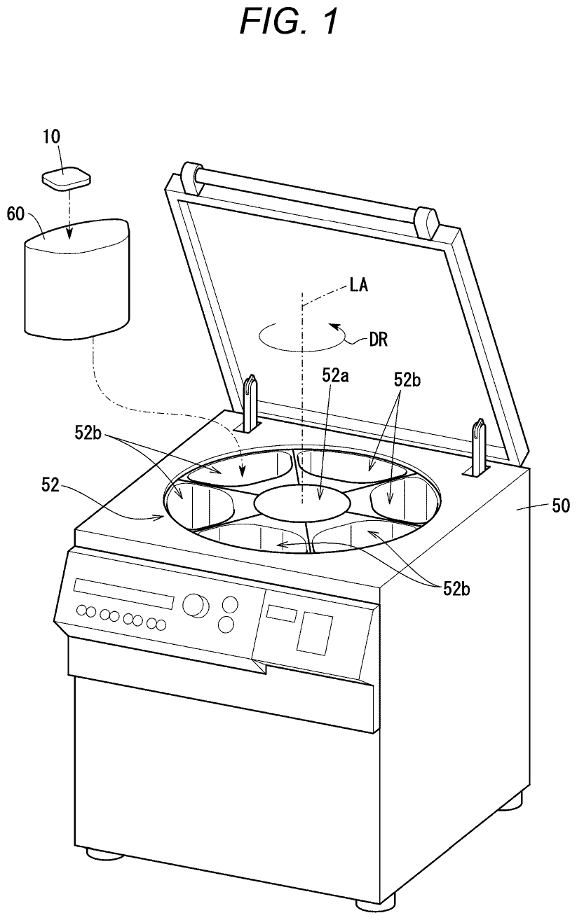

A filter device according to at least one embodiment of the present disclosure will be described with reference to the drawings. FIG. 1 is a perspective view illustrating a centrifugal separator 50 to which a filter device 10 according to a first embodiment is to be mounted. FIG. 1 illustrates the filter device 10, an insert unit 60, and a centrifugal separator 50.

The centrifugal separator 50 is a machine that centrifugally separates blood. The centrifugal separator 50 includes a centrifugal drum 52. The centrifugal drum 52 includes a central body 52a and a plurality of unit insertion portions 52b (e.g., receivers).

The plurality of unit insertion portions 52b are disposed so as to surround the central body 52a. An insert unit 60 (e.g., container, receptacle, etc.) can be inserted into each of the plurality of unit insertion portions 52b. The insert unit 60 is attached to the centrifugal separator 50 by being inserted into the corresponding unit insertion portion 52b.

A blood bag system (not illustrated) may be accommodated in the insert unit 60. The blood bag system may include a blood bag containing blood before being centrifuged. The blood contained in the blood bag is, for example, whole blood, but may be buffy coat as described in Japanese U.S. Pat. No. 5,223,006 B2 above. The buffy coat contains red blood cells, platelets, white blood cells, and the like.

The centrifugal separator 50 rotates the insert unit 60 inserted into the unit insertion portion 52b about the central body 52a. More specifically, the centrifugal separator 50 rotates the insert unit 60 inserted into the unit insertion portion 52b along a rotation direction DR about the center line of rotation (e.g., rotation center) LA illustrated in FIG. 1. Accordingly, a centrifugal force is applied to the entire insert unit 60. The blood is centrifuged by the centrifugal force. The center line of rotation LA is, for example, along a gravity direction (e.g., second direction D2 to be described later).

The filter device 10 is mounted on the insert unit 60. The filter device 10 is mounted to, for example, but not limited to, an upper part of the insert unit 60.

The filter device 10 is mounted to the insert unit 60, and thus is mounted to the centrifugal separator 50 via the insert unit 60. The centrifugal force described above is applied not only to the insert unit 60 but also to the filter device 10. Note that the filter device 10 may be directly mounted to the centrifugal separator 50.

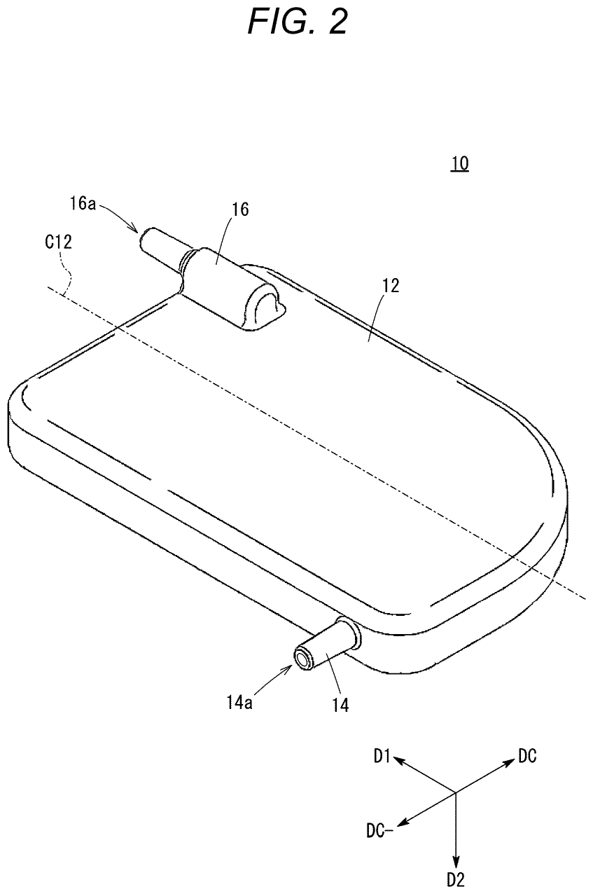

FIG. 2 is a perspective view illustrating the filter device 10 in accordance with embodiments of the present disclosure.

As described above, the filter device 10 may be mounted to the centrifugal separator 50. In the following, the configuration of the filter device 10 will be described based on the premise that the filter device 10 is mounted to the centrifugal separator 50.

As illustrated in FIG. 2, reference may be made to a centrifugal direction DC, a centripetal direction DC−, a first direction D1, and a second direction D2. The centrifugal direction DC is a direction of the centrifugal force applied to the filter device 10 by the centrifugal separator 50. The first direction D1 is a direction of a tangential velocity in the circular movement of the filter device 10 by the centrifugal separator 50. The first direction D1 is orthogonal to (e.g., intersects) the centrifugal direction DC. In some embodiments, the first direction D1 may be arranged perpendicular to the centrifugal direction DC. The second direction D2 is a gravity direction. In the present embodiment, the centrifugal direction DC and the first direction D1 are orthogonal to the second direction D2. The centripetal direction DC− is a direction opposite to the centrifugal direction DC.

The filter device 10 includes a housing 12. The housing 12 can be formed in, for example, a box shape. In the present embodiment, the thickness direction of the housing 12 coincides with the second direction D2. Stated another way, the thickness of the housing 12 may be measured as a dimension taken along the second direction D2. A length of the housing 12 may be measured as a dimension in the first direction D1, and a width of the housing 12 may be measured as a dimension in the centrifugal direction DC and/or centripetal direction DC−.

The housing 12 includes an inlet portion 14 and an outlet portion 16. The inlet portion 14 has an inlet port 14a. The outlet portion 16 has an outlet port 16a. Each of the inlet port 14a and the outlet port 16a is an opening that establishes communication between the inside and the outside of the housing 12. Stated another way, the inlet port 14a is fluidly connected to the outlet port 16a via at least one passage inside the housing 12. The inlet portion 14 and the outlet portion 16 illustrated in FIG. 2 protrude from the housing 12, but are not limited thereto.

The inlet port 14a is located between a center line C12 of the housing 12 along the first direction D1 and the rotation center LA (see, e.g., FIG. 1) of the centrifugal separator 50. On the other hand, the outlet port 16a is located in the centrifugal direction DC relative to the center line C12 of the housing 12.

Different blood bags may be connected to the inlet portion 14 and the outlet portion 16. The blood bag connected to the inlet portion 14 is a blood bag containing, in advance, blood before being centrifuged. The blood bag connected to the outlet portion 16 is a blood bag for accommodating blood (e.g., a blood component) that has passed through the filter device 10.

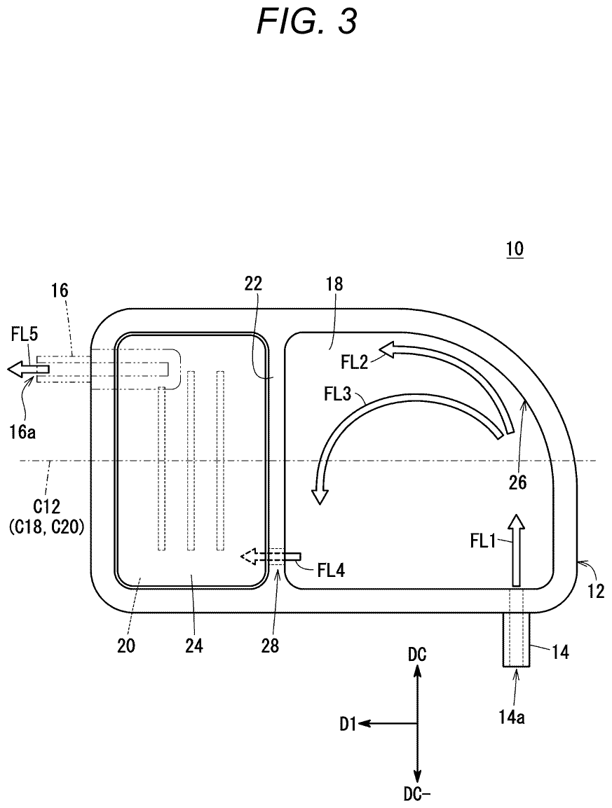

FIG. 3 is a plan view illustrating an internal structure of the filter device 10 in accordance with embodiments of the present disclosure.

The housing 12 further includes a first space 18 (e.g., first interior space), a second space 20 (e.g., a second interior space), a partition wall 22, a white-blood-cell removal filter 24, and a communication port 28. The first space 18, the second space 20, the partition wall 22, and the white-blood-cell removal filter 24 are provided in the housing 12. The first space 18 and the second space 20 are separated by the partition wall 22. The first space 18 may be fluidly interconnected to the second space 20 by the communication port 28, which passes through the partition wall 22.

The first space 18 and the second space 20 are arranged in the first direction D1. The second space 20 is located in the first direction D1 relative to the first space 18. For instance, the first space 18 and second space 20 may be arranged offset a distance from one another (e.g., side-by-side) in the first direction D1 separated by the partition wall 22.

The inlet port 14a is connected (e.g., fluidly interconnected) to the first space 18. Therefore, blood flows into the first space 18 from the outside of the housing 12 through the inlet port 14a (FL1).

The inlet port 14a is positioned between a center line C18 of the first space 18 and the rotation center LA of the centrifugal separator 50. The center line C18 of the first space 18 extends in the first direction D1. More specifically, the inlet port 14a is located on the side (e.g., of the center line C18) in the centripetal direction DC− in the first space 18.

In the present embodiment, the center line C12 of the housing 12 described above coincides with the center line C18 of the first space 18. Therefore, the inlet port 14a is located between the center line C12 of the housing 12 and the rotation center LA of the centrifugal separator 50.

The inlet port 14a may be formed along the centrifugal direction DC. In this case, the blood smoothly flows through the inlet port 14a and flows into the first space 18 by the centrifugal force applied by the centrifugal separator 50.

The first space 18 has a curved wall 26 (e.g., curved interior wall). The curved wall 26 is a part of the inner wall defining the first space 18. The curved wall 26 is located in the centrifugal direction DC relative to the center line C18 (C12) of the first space 18. The curved wall 26 is located on one side of the first space 18 in the first direction D1.

The curved wall 26 is curved so as to guide blood (e.g., entering the first space 18 from the inlet port 14a, etc.) to the other side of the first space 18 (e.g., opposite the curved wall 26, etc.) in the first direction D1. As a result, the blood can flow along the curved wall 26 without flowing back toward the inlet port 14a (FL2, FL3). Note that the partition wall 22 described above is located on the other side of the first space 18 (e.g., opposite the curved wall 26) in the first direction D1.

The blood flowing into the first space 18 is centrifuged by the centrifugal separator 50. As a result, the blood in the first space 18 has a layer of a supernatant liquid and a layer of a sedimentation liquid. The layer of the sedimentation liquid is located in the centrifugal direction DC relative to the layer of the supernatant liquid.

The main component of the sedimentation liquid is white blood cells. The reason why white blood cells are considered to be the main component of the sedimentation liquid is that white blood cells are relatively heavy blood components. That is, white blood cells are blood components that are relatively easily precipitated, and thus, are main components of the sedimentation liquid. On the other hand, the supernatant liquid contains a blood component lighter than white blood cells. For example, the supernatant liquid contains large quantities of platelets and the like.

In this manner, the white blood cells contained in the blood flowing into the first space 18 are collected on the side of the first space 18 in the centrifugal direction DC (e.g., above the center line C18 in FIG. 3) by the centrifugal force applied by the centrifugal separator 50 (FL2). On the other hand, blood components other than white blood cells are collected on the side of the first space 18 in the centripetal direction DC− (FL3) (e.g., below the center line C18 in FIG. 3).

The communication port 28 is an opening that establishes communication (e.g., fluid communication, etc.) between the first space 18 and the second space 20. The communication port 28 is located between the center line C18 (C12) and the rotation center LA of the centrifugal separator 50. More specifically, the communication port 28 is located in the centripetal direction DC− in the first space 18 (e.g., below the center line C18 in FIG. 3). The communication port 28 is formed in the partition wall 22, for example, but is not limited thereto.

At least a portion of the blood flowing into the first space 18 flows into the second space 20 through the communication port 28 (FL4). As described above, blood components other than white blood cells are collected on the side of the first space 18 in the centripetal direction DC−. Therefore, blood components other than white blood cells easily flow into the second space 20 through the communication port 28. In other words, white blood cells are less likely to flow into the second space 20 than the other components in the blood.

The inlet port 14a is located on one side of the first space 18 in the first direction D1 (e.g., the right-hand side of the first space 18 shown in FIG. 3). On the other hand, the communication port 28 is located on the other side of the first space 18 in the first direction D1 (e.g., the left-hand side of the first space 18 shown in FIG. 3). With this configuration, it is possible to prevent the blood flowing by the centrifugal force from immediately flowing from the inlet port 14a to the communication port 28. That is, it is possible to prevent the blood flowing in through the inlet port 14a from reaching the communication port 28 before being separated into the above-described sedimentation liquid and supernatant liquid.

In the second space 20, the above-described white-blood-cell removal filter 24 is disposed. The white-blood-cell removal filter 24 includes a filter medium for removing white blood cells from blood. The blood that has flown into the second space 20 is filtered by the white-blood-cell removal filter 24. Thus, even if white blood cells flow into the second space 20, the white blood cells are removed by the white-blood-cell removal filter 24.

The second space 20 is provided with the outlet portion 16. The outlet portion 16 is provided with the outlet port 16a. The outlet port 16a is in fluid communication with the second space 20. Therefore, the blood (e.g., blood without white blood cells, etc.) in the second space 20 can flow out of the housing 12 through the outlet port 16a (FL5).

The outlet port 16a is located in the centrifugal direction DC relative to a center line C20 (e.g., above the center line C20 in FIG. 3) of the second space 20 extending in the first direction D1. On the other hand, the communication port 28 is located in the centripetal direction DC-relative to the center line C20 of the second space 20 (e.g., below the center line C20 in FIG. 3) extending in the first direction D1. Since the positional relationship between the outlet port 16a and the communication port 28 is as described above, the blood flowing into the second space 20 easily reaches the outlet port 16a by the centrifugal force applied by the centrifugal separator 50. That is, the blood can be smoothly taken out from the outlet port 16a, for example, only by operating the centrifugal separator 50. In the present embodiment, the center line C12 of the housing 12 described above coincides with the center line C20 of the second space 20.

The communication port 28 is located on one side of the second space 20 in the first direction D1 (e.g., the right-hand side of the second space 20 shown in FIG. 3). On the other hand, the outlet port 16a is located on the other side of the second space 20 in the first direction D1 (e.g., the left-hand side of the second space 20 shown in FIG. 3). Since the positional relationship between the communication port 28 and the outlet port 16a is as described above, a path long enough to pass the blood can be formed in the white-blood-cell removal filter 24. This configuration makes it possible to more reliably remove white blood cells by the white-blood-cell removal filter 24.

As described above, according to the present embodiment, the blood flowing into the first space 18 is centrifuged by the centrifugal separator 50. Thus, the amount of white blood cells contained in the blood flowing into the second space 20 is reduced. The white blood cells remaining in the blood flowing into the second space 20 are removed by the white-blood-cell removal filter 24. That is, according to the present embodiment, the removal of white blood cells by centrifugation and the removal of white blood cells by the white-blood-cell removal filter 24 can be achieved on blood only by operating the centrifugal separator 50.

Modifications according to the above embodiment will be described below. Note that the description overlapping with the above embodiment will be appropriately omitted. Elements described in the above embodiment are denoted by the same reference numerals as those in the above embodiment unless otherwise specified.

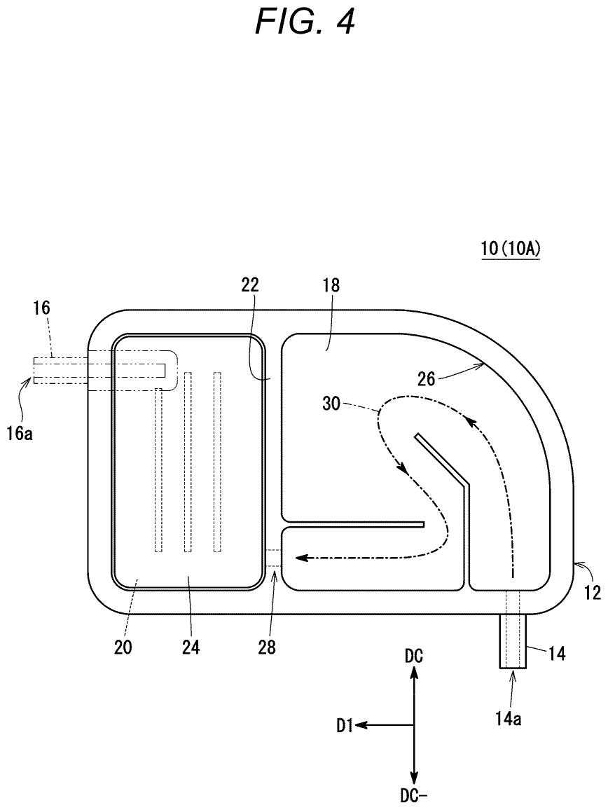

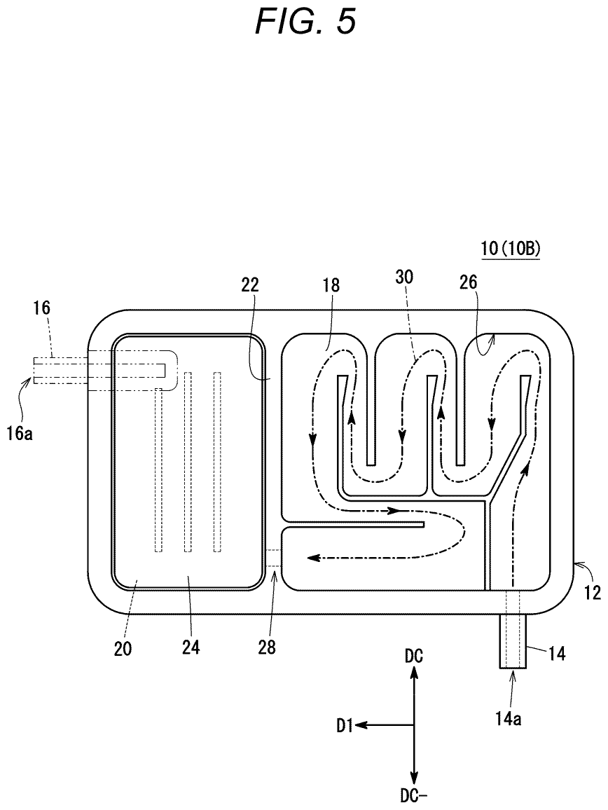

FIG. 4 is a plan view illustrating an internal structure of a filter device 10 (10A) according to a first modification. FIG. 5 is a plan view illustrating an internal structure of a filter device 10 (10B) according to a second modification.

As illustrated in FIGS. 4 and 5, a meandering flow channel 30 for allowing blood flowing in through the inlet port 14a to meander may be formed in a first space 18. The blood flowing in through the inlet port 14a reaches the communication port 28 through, or by way of, the meandering flow channel 30. Thus, it is possible to more reliably prevent the blood from immediately flowing into the communication port 28 from the inlet port 14a. That is, it is possible to more reliably prevent blood from which white blood cells have not been removed by centrifugation from reaching the second space 20. The specific shape of the meandering flow channel 30 is not limited to the examples in FIGS. 4 and 5.

Note that the present invention is not limited to the above disclosure and can take various configurations without departing from the gist of the present invention.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A filter device to be mounted on a centrifugal separator, the filter device comprising:
a first space which is formed in a housing and into which blood flows through an inlet port;
a second space which is formed in the housing and from which the blood flows out through an outlet port;
a partition wall that separates the first space and the second space;
a communication port that establishes communication between the first space and the second space; and
a white-blood-cell removal filter that is disposed in the second space and that removes a white blood cell contained in the blood, wherein:
the first space and the second space are arranged in a first direction intersecting a centrifugal direction in which a centrifugal force is applied, and
the inlet port and the communication port are located between a center line of the housing along the first direction and a rotation center of the centrifugal separator.

2. The filter device of claim 1, wherein the outlet port is located in the centrifugal direction relative to the center line.

3. The filter device of claim 1, wherein the inlet port is located on one side of the first space in the first direction, and wherein the communication port is located on another side of the first space in the first direction.

4. The filter device of claim 3, wherein the first space includes a curved wall, and wherein the curved wall is located in the centrifugal direction relative to the center line and located on the one side of the first space.

5. The filter device of claim 1, wherein the communication port is located on one side of the second space in the first direction, and wherein the outlet port is located on another side of the second space in the first direction.

6. The filter device of claim 1, wherein the first space is provided with a meandering flow channel for allowing the blood flowing in through the inlet port to meander.

7. A filter device, comprising:
a housing comprising a length extending along a first direction from a first end of the filter device to a second end of the filter device, the housing comprising a width extending along a second direction from a first side of the filter device to a second side of the filter device, wherein a center of the width defines a center line extending along the first direction bisecting the width between the first side of the filter device and the second side of the filter device, and wherein the housing further comprises:
a first space disposed inside the housing adjacent the first end of the filter device, the first space spanning across the center line, wherein the first space comprises a curved interior wall arranged adjacent the second side of the filter device;
a second space disposed inside the housing adjacent the second end of the filter device, the second space spanning across the center line;
a partition wall arranged between the first space and the second space, wherein the partition wall separates the first space from the second space;
an inlet port arranged on the first side of the filter device, wherein the inlet port is in fluid communication with the first space, and wherein the inlet port comprises an inlet flow direction extending toward the curved interior wall of the first space;
a communication port arranged in the partition wall, wherein the communication port fluidly interconnects the first space and the second space; and
an outlet port arranged on the second side of the filter device, wherein the outlet port is in fluid communication with the second space;
wherein, when a centrifugal force is applied to the filter device in the second direction, a blood flow path is defined between the inlet port, the curved interior wall, the communication port, and the outlet port.

8. The filter device of claim 7, wherein the inlet flow direction extends into the first space along the second direction.

9. The filter device of claim 8, wherein the outlet port comprises an outlet flow direction that extends out of the second space along the first direction.

10. The filter device of claim 9, wherein the communication port comprises a communication flow direction that extends from the first space into the second space along the first direction.

11. The filter device of claim 10, wherein the outlet port is arranged adjacent the second end of the filter device.

12. The filter device of claim 11, wherein the communication port is arranged closer to the first side of the filter device than the second side of the filter device.

13. The filter device of claim 12, wherein the curved interior wall provides a smooth curved flow transition in the blood flow path at a first wall portion in the first space that is arranged parallel to the second direction to a second wall portion of the first space that is arranged parallel to the first direction.

14. The filter device of claim 13, wherein the first wall portion is arranged on the first side of the filter device adjacent the first end of the filter device.

15. The filter device of claim 14, wherein the second wall portion is arranged on the second side of the filter device adjacent the partition wall.

16. The filter device of claim 15, wherein a majority of the curved interior wall is arranged on the second side of the filter device.

17. The filter device of claim 7, further comprising:
a white-blood-cell removal filter disposed inside the second space, wherein the white-blood-cell removal filter comprises a filter medium that is configured to remove white blood cells from a portion of blood flowing therethrough.

18. The filter device of claim 7, wherein the first space further comprises a meandering flow channel that changes a direction of blood flowing along the blood flow path inside the first space before reaching the communication port.

19. A filter device, comprising:
a housing, comprising:
a first interior space formed inside the housing, the first interior space comprising an inlet port arranged in fluid communication with the first interior space and a curved interior wall arranged opposite the inlet port;
a second interior space formed inside the housing, the second interior space comprising an outlet port arranged in fluid communication with the second interior space;
a partition wall arranged between the first interior space and the second interior space, wherein the partition wall separates the first interior space from the second interior space; and
a communication port disposed in the partition wall, the communication port arranged in fluid communication with the first interior space and the second interior space;
wherein the first interior space and the second interior space are arranged side-by-side in a first direction that is arranged perpendicular to a centrifugal direction, wherein, when a centrifugal force is applied to the filter device in the centrifugal direction, a blood flow path is defined between the inlet port, the curved interior wall, the communication port, and the outlet port.

20. The filter device of claim 19, further comprising:
a white-blood-cell removal filter disposed inside the second interior space, wherein the white-blood-cell removal filter comprises a filter medium that is configured to remove white blood cells from a portion of blood flowing through the white-blood-cell removal filter.
